(19) United States
(12) Patent Application Publication
(10) Pub. No.: US 2004/0086867 A1
(43) Pub. Date: May 6, 2004

(54) Method for detecting nucleic acid

(76) Inventor: Jian Han, Huntsville, AL (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/284,656

(22) Filed: Oct. 30, 2002

(51) Int. Cl. C12Q 1/68

(52) U.S. Cl. 435/6

(57) ABSTRACT

Disclosed is a new methodology for detecting and/or quantitating nucleic acid sequences, termed “Reporter Oligo Capturing After Specific Hybridization,” (ROCASH). As described above, the prior art methods for detection of nucleic acid samples are often forced to trade sensitivity in the detection assay to achieve increased specificity. In this method, unlike the prior art methods, the specificity of hybridization and the sensitivity of the detection step are provided by different nucleic acid sequences hybridizing in different steps of the method. Therefore, conditions for these critical steps may be optimized independently of each other.

### FIELD OF THE DISCLOSURE

The present disclosure concerns methods for detecting and quantitating nucleic acids in a sample.

### BACKGROUND

The present disclosure represents an advance in the art of detecting nucleic acid sequences in a sample. Prior methods of detecting nucleic acid sequences in a sample suffered from the drawback that the same probe sequences that generated specificity in the detection reaction were also responsible for generating sensitivity in the reaction. This was so because the same nucleic acid sequences responsible for generating both the specificity and sensitivity of detection. As a result, any change in the reaction that impacted specificity also impacted sensitivity, and vice versa. In addition, many prior art methods depended on enzymatic reactions to generate specificity (such as allele specific PCR, allele specific primer extension, or allele specific ligation) and were therefore, limited by the efficiency of the enzymatic reaction. The use of enzymatic reactions to generate specificity makes the assays more complicated, decreasing throughput and increasing the probability of errors. These techniques are summarized in “Accessing Genetic Variation” Genotyping Single Nucleotide Polymorphisms”, Ann-Christine Syvänen, Nature Reviews Genetics, December 2001, Vol.2, p930-942

The detection of specific nucleotide sequences is becoming ever important. Such detection methods can be used to diagnose the presence of certain disease states caused by the quantitative or qualitative changes of genetic material (such as gene expression pattern changes in cancer, point mutations in genetic diseases, and the presence of infectious agents), can be used to predict a predisposition to a given disease state and to aid in the identification and isolation of candidate causative genes for disease states.

### SUMMARY

The present disclosure describes a new methodology for detecting and/or quantitating nucleic acid sequences in a sample. The new method is especially useful for the detection and/or quantification of multiple nucleic acid sequences in a sample. The method is termed “Reporter Oligo Capturing After Specific Hybridization,” or ROCASH for short, As described above, the prior art methods for detection of nucleic acid samples are often forced to trade sensitivity in the detection assay to achieve increased specificity. This is a necessary tradeoff in the prior art methods since specificity of the hybridization step between the target and the reporter, and sensitivity of the detection of the reporter are provided by the same nucleic acid sequences or the same step in the detection assay. In the ROCASH method, unlike the prior art methods, the specificity of hybridization and the sensitivity of the detection step are provided by different nucleic acid sequences hybridizing in different steps of the method. Therefore, conditions for these critical steps may be optimized independently of each other.

In general, in one embodiment of the ROCASH method each set of reporter oligonucleotides is designed to bind specifically to a sequence of interest in a nucleic acid source (the target nucleic acid, or the target). A nucleic acid source is provided containing the target nucleic acid. The nucleic acid source and the target nucleic acid may be derived from any organism, including, but not limited to, humans. This target nucleic acid may be amplified by standard techniques, such as PCR. Alternatively, a nucleic acid source containing target nucleic acid may be used without any amplification.

In one embodiment where amplification of the target nucleic acid is used, the amplification may incorporate a tag means at the 5′ end of the primer in order to purify or enrich the amplification products containing the target nucleic acids. The tag means may be a nucleic acid sequence, an amino acid sequence or a chemical moiety (for example biotin) or other moiety capable of binding to a purification means. The selection of the purification means will depend on the selction of the tag means and is within the skill of one in the art. This tag means and purification means will aid in the separation of the amplification products containing the target nucleic acid from un-wanted strands and un-used primers and nucleotides. In one embodiment, the amplification may be accomplished by PCR.

After amplification (if used), the reverse strands are denatured from the template strands. Using the 5′ tag means and purification means, the reverse strands are separated from the template strand. Such separation methods will vary depending on the tag means and purification means incorporated into the PCR amplification products. The selection of such means and separation methods are within the ordinary skill in the art. An example is provided below, using biotin as the tag means and streptavidin conjugated magnetic beads as the purification means. However, purification of the reverse strands is optional.

The reporter oligonucleotides are then added to the target nucleic acid mixture. The reporter oligonucleotides bind the target nucleic acid contained in the reverse strands through the hybridization domain. The mixture is washed to remove reporter oligonucleotides that have not bound the target nucleic acid. The reporter oligonucleotides/target nucleic acid complex is then denatured, releasing the bound reporter oligonucleotides. The target nucleic acid is subsequently removed with the aid of the incorporated tag or by other methods. Once again such removal methods will vary depending on the tag incorporated into the PCR amplification products and are within the ordinary skill in the art.

A collecting means is mixed with the reporter oligonucleotides left in solution. The collecting means comprises a complementary sequence to a selected domain of the reporter oligonucleotide that is specific for the reporter oligonucleotide and a second detection means. The collecting means removes a substantial portion of the free reporter oligonucleotides from solution. The substantial portion of reporter oligonucleotide removed is sufficient to generate a detectable signal in the ROCASH assay and will vary depending on the concentrations of reagents used and other conditions. The amount of reporter oligonucleotide removed may be adjusted as determined by one of ordinary skill in the art. The collecting means with bound reporter oligonucleotides is then analyzed to determine the amount or reporter oligonucleotide that bound to the target nucleic acid and to determine the identity of the target nucleic acid. In this method, the amount of reporter oligonucleotide that is detected bound to the collecting means is proportional to the amount of the target nucleic acid present in the sample. Therefore, the amount of target nucleic acid present in a sample can be determined by measuring the levels of reporter oligonucleotides.

In one embodiment, the new detection methodology is particularly useful in the detection of single nucleotide polymorphisms (SNPs) and similarly, base change mutations at DNA level. The ROCASH method is particularly well suited to the identification of multiple SNP's in a single reaction because the specificity of the hybridization reactions can be varied, while the conditions that define the sensitivity of the detection step can be maintained as a constant.

In an alternate embodiment, the ROCASH method can be used to quanititatively detect gene expression levels at mRNA level. Using the methodology described herein, the expression levels of at least 100 genes could be simultaneously detected in a single assay, either with or without amplification of the mRNA sequences.

In yet another embodiment, the ROCASH method can be used to detect differences in gene dosage that may be associated with increases or decreases in genomic DNA copy numbers.

In another embodiment, the ROCASH method can be used for nucleotide analysis in PCR reaction products and be used for the detection of infectious agents.

### BRIEF DESCRIPTION OF THE FIGURES

FIG. 1A shows one embodiment of the reporter oligonucleotide of the present disclosure.

FIG. 1B shows one embodiment of the collecting means of the present disclosure.

FIG. 2 shows one embodiment of the ROCASH method of the present disclosure.

### DETAILED DESCRIPTION

The present disclosure describes a method for detecting and/or quantitating nucleic acid sequences in a sample. Nucleic acids are defined as DNA, RNA, or synthetic or semi-synthetic derivatives or combinations thereof, which include, but are not limited to, genomic DNA, mRNA, cDNA and PCR products. As discussed above, the ROCASH method separates the specificity step and the sensitivity step in the detection of nucleic acid sequences in a sample. The separation of specificity and sensitivity is made possible, in part, by the design of the ROCASH reporter oligonucleotide when used in combination with the methods presented herein.

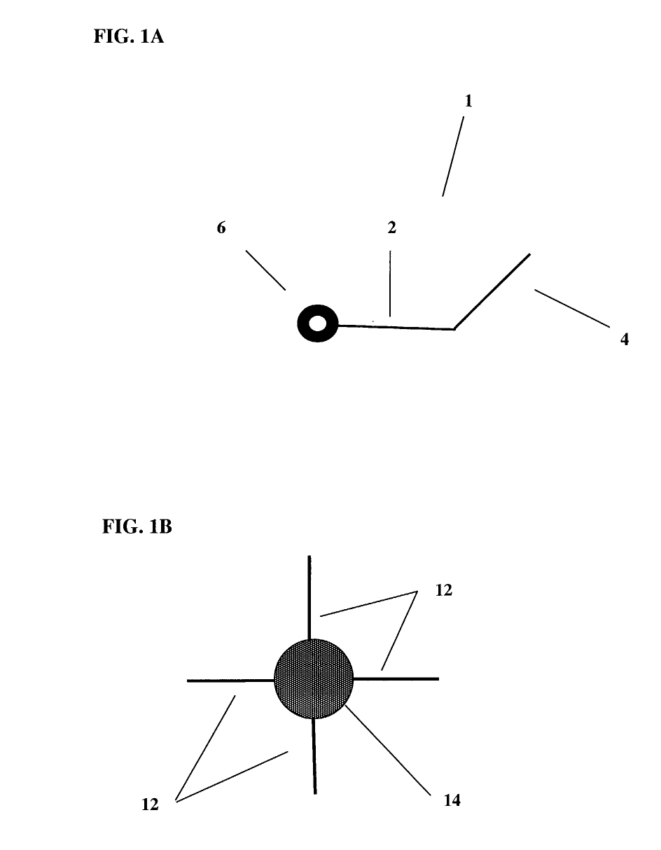

The reporter oligonucleotide has three separate functional domains as illustrated in FIG. 1: 1) the hybridization domain 2, 2) the region tag 4, and 3) the first detecting means 6.

The hybridization domains 2 of the reporter oligonucleotides 1 are designed to bind specifically to a target nucleic acid. The design of the hybridization domain can be any that will allow specific detection with an acceptable background as guided by principles known to one of ordinary skill in the art in the field. Since the hybridization domain is specifically engineered to hybridize to the target nucleic acid, the specificity with which the hybridization domain binds the target nucleic acid can be easily adjusted. For instance, the length of the hybridization domain can be adjusted to alter the specificity with which the hybridization domain binds the target nucleic acid. In general, for detecting single nucleotide polymorphisms or mutations, a shorter hybridization domain will give increased specificity and allelic differentiation power as a mismatch between the target nucleic acid and the hybridization domain will have a significant impact on the hybridization efficiency of the hybridization domain to the target nucleic acid. However, if the hybridization domain is too short, the sensitivity of the detection assay will suffer as a result of lowered hybridization efficiency of the hybridization domain to the target nucleic acid. One of ordinary skill in the art would be able to alter the parameters of the hybridization domain to achieve the desired specificity of binding of the hybridization domain to the target nucleic acid.

The region tag 4 is designed to provide a means to differentiate the individual set of reporter oligonucleotides 1 from one another. Each region tag 4 specifically binds a complementary sequence on the collecting means 10, which is termed the complementary region tag, or cRT, (labeled 12 in FIG. 1B) (as discussed generally above and in greater detail below). In one embodiment, a unique region tag 4 is associated with each hybridization domain 2 on each reporter oligonucleotide 1. In an alternate embodiment the same region tag 4 may be associated with different hybridization domains 2. In either case, the identity of each hybridization domain associated with ach region tag is known. The cRTs 12 are be designed such that they is capable of binding to the region tags 4 of the respective reporter oligonucleotides 1 with essentially equal kinetics and binding properties (as discussed below). In one embodiment, the region tag 4 may be a nucleic acid sequence and the cRT 12 may be a complementary nucleic acid sequence. In alternate embodiments, the region tag 4 may be a first chemical moiety (organic or inorganic) that is capable of specifically bonding to a second chemical moiety (organic or inorganic) serving as the cRT 12. In another embodiment, the region tag 4 may be a first moiety that is capable of specifically interacting with an amino acid sequence serving as the cRT 12. In yet another embodiment, the region tag 4 may be an amino acid sequence capable of binding to a second amino acid sequence serving as the cRT 12.

In one embodiment, the region tags 4 and the cRTs 12 are nucleic acid sequences. In one embodiment, the region tags 4 are designed to be about 20 nucleotides in length. Briefly, the region tags 4 are a series nucleic acid sequences are randomly generated using a computer algorithm. The GC % content of the sequences is at least 60%, in one embodiment. Higher GC % content may be selected if desired. As a result, each of the region tags 4 will have identical Tm when hybridized to their complementary cRT 12. To reduce the chances of the sequences of the region tags and the cRTs from appearing in human genome at high frequency (which will generate high background), each of the region tags has at least one CpG dinucleotides and at least one TpA dinucleotides (as CpG and TpA are the lest frequent dimmers in the human genome). The region tags and cRTs are BLAST searched using the standard search tools against the human genome to exclude those have high frequency or degree of homology to other human sequences. The nucleic acid sequences are then examined to search for hairpin loop structures and other structure that may disrupt binding. As a final step, the remaining region tags are selected so that there is no significant homology among themselves.

As used in the present disclosure, the use of the region tags 4 and the complementary cRTs 12 provides a universal starting point for optimizing the sensitivity of the detection reaction, regardless of the sequences being detected. In the embodiment above, the conditions for the hybridization of each of the region tags 4 to their cRTs 12 are essentially the same.

The third domain of the reporter oligonucleotide 1 comprises a first detection means 6. The first detection means 6 may be any means that is capable of producing a first signal when placed in an appropriate apparatus for detection and analysis. In one embodiment, the first detection means 6 produces a first signal that is fluorescent. The selection of the appropriate fluorescent label for incorporation into the reporter oligonucleotide 1 will depend on the analysis method to detect the fluorescent label. In one embodiment, the first detection means is a PE label. In one embodiment, the reporter oligonucleotide 1 incorporates a biotin moiety opposite the region tag as illustrated in FIG. 1A. The PE is added as a streptavidin-PE conjugate. In an alternate embodiment, the PE label may be conjugate directly to the reporter oligonucleotide. In another embodiment, the first detection means is the Cy3 label, which may be added as discussed above for the PE label.

As stated above, the cRTs 12 are coupled to a collecting means 10. The collecting means 10 also incorporates a second detection means 14. The second detection means 14 may be any means that is capable of producing a second signal when placed in an appropriate apparatus for detection and analysis. In one embodiment, the second detection means 14 produces a second signal that is fluorescent. The second detection means 14 and second signal allow the identity of the reporter oligonucleotide 1 (and therefore, the nucleic acid target) bound to the collecting means to be determined. In other words, by examining the second detection means 14, either alone or in conjunction with the first detecting means 6, a user may determine: 1) whether a reporter oligonucleotide 1 has bound the cRT 12; and 2) since the second detection means 14 is with a known cRT 12, and every cRT 12 is associated with a known region tag 4, and every region tag 4 is associated with a known hybridization domain 2, and every hybridization domain 2 is designed to hybridize with a known target, the identity of the target may be determined.

For example, the second detection means 14 may produce a detectable physical signal when placed in an appropriate apparatus for detection and analysis. Physical signal may be a color change, an emission of a given wavelength of light upon excitation or a change in the electrical properties, such as conductivity, or a change in the electromagnetic or chemical properties. Other physical signals may also be used. In another embodiment, the second detection means 14 may be spatial in nature, such as location on a solid support. For example, the cRTs 12 may be spatially resolved on the collecting means 10 (such as a chip or other solid support) such that when the reporter oligonucleotide binds a cRT 12 via the region tag 4, the first detecting means 6 is observed at a specific location, thus allowing the identity of the target nucleic acid can be determined.

In one embodiment, the first detection means 6 is a PE-streptavidin moiety and the second detection means 14 is the micropsheres used in the x-MAP™ technology from Luminex Corporation (Austin, Tex.) (described in U.S. Pat. Nos. 5,981,180 and 6,268,222). The x-MAP™ technology uses a plurality of internally color coded, spectrally addressable polystyrene microspheres. These microspheres have multiples copies of the cRT 12 attached to the outer surface. When the microspheres are used as the second detection means 14, a plurality of cRT 12 are attached to the outer surface of the microspheres. The cRTs 12 will then bind the reporter oligonucleotides 1 via the region tags 4. Since each cRT 12 is specific for only 1 region tag 4, and each region tag 4 is associated with a known hybridization domain 2, and each hybridization domain 2 is associated with a known target nucleic acid, each target nucleic acid is associated with a color coded-microsphere (as the second detection means 14).

The presence of the first detection means 6 on the reporter oligonucleotides will allow the determination of whether the reporter oligonucleotide 1 is present, while the color coded microsphere (the second detection means 14 in this embodiment) will allow the identification of the nucleic acid target. As a result, the presence and/or amount of the target nucleic acid can be determined.

The same principles apply when other collecting means are used. For example, in an alternate embodiment the collecting means 10 may be a silicon chip. On the chip, cRTs 12 are placed on the chip in predetermined location. The reporter oligonucleotides 1 will bind the cRTs 12 as described above, with each target nucleic acid being associated with a known location on the chip (as described above). As above, the first detection means 6 on the reporter oligonucleotide 1 will allow the determination of whether the reporter oligonucleotide 1 has bound the cRT 12. The identity of the target nucleic acid is determined by the location (the second detection means 14 in this embodiment) of the first detection means 6.

The first 6 and second 14 detection means produce a first and a second signal, respectively, during the detection and analysis process. The first and second signals are such that each can be discretely detected in the presence of the other. In one embodiment, the first and second signals are discrete wavelengths of light produced when the first and second detection means are excited by the appropriate wavelengths of light. In an alternate embodiment, the first signal is a discrete wavelength of light produced when the first detection means is excited and the second detection means is a physical property, such as, but not limited to, position, electrical properties, electromagnetic properties and chemical properties.

The ROCASH method may be used to detect and/or identify a single target nucleic acid or multiple target nucleic acids. When used to detect and/or identify multiple target nucleic acids, multiple sets of reporter oligonucleotides and or multiple sets of collecting means are used. A set of reporter oligonucleotides is defined as a reporter oligonucleotide comprising a hybridization domain specific for a known target nucleic acid, with the hybridization domain being associated with a unique region tag. A set of collecting means is defined as a collecting means comprising a plurality of complementary region tags that hybridize to region tags of the reporter oligonucleotides such that a single target nucleic acid or an analysis group of target nucleic acids are associated with a known second signal generated by the collecting means. An analysis group is defined as any set of target nucleic acids that are intended to be analyzed as a group. An analysis group may include, but is not limited to, multiple target nucleic acids from a single gene, single or multiple target nucleic acids from a family or related genes, single or multiple target nucleic acids from genes involved in a common cellular pathway and/or single or multiple target nucleic acids from genes involved in common cellular mechanisms.

The hybridization conditions between the hybridization domain of the reporter oligonucleotide and the target nucleic acid and the region tags and the complementary region tags should be selected such that the specific recognition interaction, i.e., hybridization, of the two is both sufficiently specific and sufficiently stable (Hames and Higgins (1985) Nucleic Acid Hybridisation: A Practical Approach, IRL Press, Oxford). The hybridization conditions will usually be selected to be sufficiently specific such that the fidelity of base matching will be properly discriminated. Of course, control hybridizations should be included to determine the stringency and kinetics of hybridization. These conditions will be dependent both on the specific sequence and often on the guanine and cytosine (GC) content of the complementary hybrid strands. For binding between target nucleic acids and hybridization domains, the conditions may be selected to be universally equally stable independent of the specific sequences involved, while still maintaining specificity. This may make use of a reagent such as an alkylammonium buffer. An alkylammonium buffer tends to minimize differences in hybridization rate and stability due to GC content. By virtue of the fact that sequences then hybridize with approximately equal affinity and stability, there is relatively little bias in strength or kinetics of binding for particular sequences. Temperature and salt conditions along with other buffer parameters should be selected such that the kinetics of renaturation should be essentially independent of the specific target subsequence or oligonucleotide probe involved. For the region tag complementary region tag interactions, these conditions exist by virtue of the design of these components.

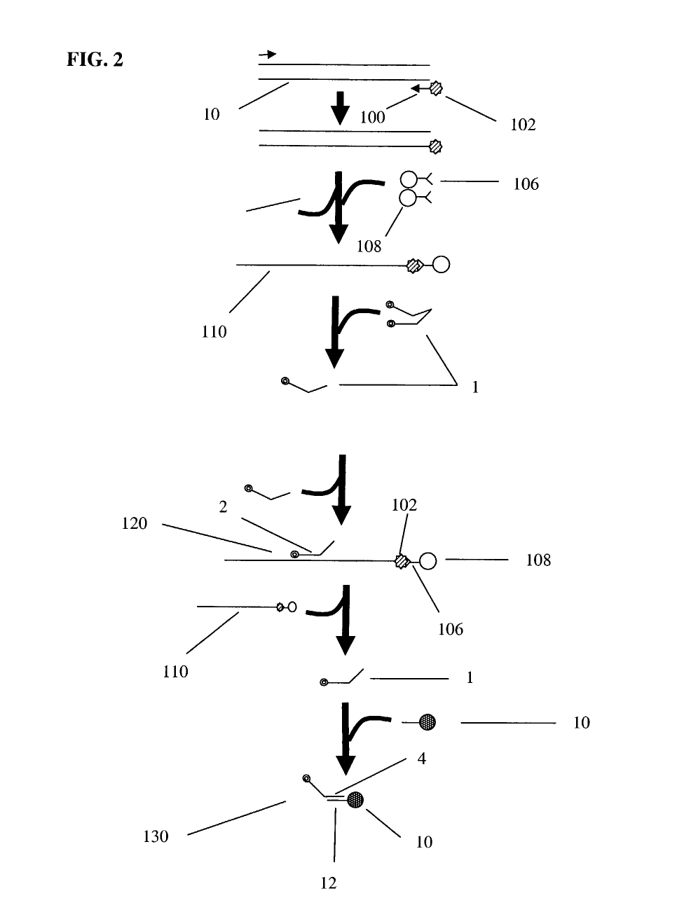

One embodiment of the ROCASH method is shown in FIG. 2. Using the ROCASH detection methodology, increased specificity in the detection can be obtained without sacrificing the sensitivity of detection. This is due, in part, because the reporter oligonucleotide 1 used in the ROCASH method has 2 separate functional domains that undergo discrete hybridization reactions: 1) the hybridization domain 2 which hybridizes to the target nucleic acid (which affects specificity of detection); and 2) the region tag 4 which hybridizes to the cRT 12 of the collecting means 10 (which affects the sensitivity of detection). Since this is the case, the specificity component of the detection reaction can be modulated independently of, and therefore, separately from, the sensitivity component of the detection reaction. Both components of the detection reaction can be carried out without enzymatic reactions, which further increase the efficiency of the detection reaction.

The target nucleic acid sequence of interest is amplified by standard PCR techniques as discussed above. Any number of target nucleic acid sequences may be amplified by a multiplexed PCR reaction, and each PCR product could include many targets to be identified by different reporter oligonucleotides 1. For detection of multiple target nucleic acids in a single sample, these multiple target nucleic acid sequences can be amplified in one reaction, or the target nucleic acid sequences can be amplified in multiple reactions and combined for the detection steps described below. Design of the appropriate PCR amplification conditions and primers is within the ordinary skill in the art. The PCR primers may incorporate various tags means at their 5′ end to aid in the purification and/or enrichment steps discussed below.

In the embodiment shown in FIG. 2, a biotin molecule 102 is used as the tag means. The biotin molecule is attached to the 5′ end of the primer 100. As a result, the reverse strands 110 of the PCR reaction incorporate the biotin 102 tag means. A purification means, in this case a streptavidin molecule 106 is conjugated to a magnetic bead 108 (Dynal M270), is then added to the PCR reaction. The purification means may be added during the PCR process or after the process is completed. In an alternate embodiment, the reverse PCR primer 100 incorporates a nucleic acid sequence tag means discrete from the primer sequence that is used to amplify the target nucleic acid (located at the 5′ end of the primer). During the PCR amplification process the sequence of the tag means is incorporated into the amplified PCR products. In one embodiment, the nucleic acid sequence is that of at least a portion of the T7 promoter. A purification means, in this case a complementary nucleic acid sequence to the tag means is conjugated to a magnetic bead is added to the PCR reaction. The purification means are added to the PCR reaction and act as primers during subsequent rounds of PCR amplification (on-bead PCR). As a result, the reverse strands of the PCR amplification incorporate the purification means at their 5′ ends. Alternate labels may also be incorporated into the PCR primers, as would be known to one of ordinary skill in the art. In alternate embodiment, the nucleic acid targets do not require amplification.

After PCR amplification, the PCR products are denatured. This denaturation, and the other denaturation steps referred to in this disclosure, may occur by heating to a sufficient temperature to disrupt the DNA duplex, or by chemical means (such as, but not limited to, the addition of agents such as 5N NaOH). In most cases the reverse strands are separated from the remainder of the PCR reaction products. The method of separation will depend on the type of tag means and purification means employed. For example, if a biotin moiety is used as the tag means, then streptavidin may be used alone as the purification means, by attaching the streptavidin to a column or other support as is know in the art. Also, streptavidin may be conjugated to other moieties such as magnetic bead allowing magnetic separation using techniques standard in the art. When nucleic acids are used as the tag means, complementary nucleic acids may be used as the purification means. The complementary nucleic acids may either be used alone or conjugated to a column or other support as is known in the art, or may be conjugated to other moieties, such as magnetic beads as described above. If alternate tag means and purification means are used, alternate separation techniques may be used. Hybridization buffer (such as 1X TMAC or 1X TE) is then added to the reverse strands 110 in preparation for reporter oligonucleotide 1 hybridization to the target nucleic acid sequences.

The reporter oligonucleotides 1 are then added (at saturation concentration) to the isolated reverse strands 110. The hybridization domains 2 of the reporter oligonucleotides 1 bind the target nucleic acid of interest. There may be as many reporter oligonucleotides as there are amplified target nucleic acid sequences. The general principles relevant in the design of the hybridization domains 2 are within the ordinary skill in the art. The reporter oligonucleotide/target nucleic acid complexes 120 are then washed (such as with 1X SSC pre-warmed to 48 degrees C.) to remove reporter oligonucleotides 1 that have not bound the target nucleic acids. The tag means/purification means facilitates this separation as discussed above. The isolated reporter oligonucleotide/target nucleic acid complexes 120 are then denatured and the target nucleic acids are removed again aided by the tag means/purification means. The reporter oligonucleotides 1 that bound the target nucleic acid sequences are left in solution.

A collecting means 10 is then mixed with the reporter oligonucleotides 1. The collecting means 10 comprises a cRT (specific for the region tag 4 of the reporter oligonucleotides 1) and a second detection means 14. The cRT 12 of the collecting means 10 is designed to be complementary to the region tag 4 on the reporter oligonucleotide as discussed above. The cRT 12 then bind the region tags 4 of the reporter oligonucleotides 1. As discussed above, the individual cRTs 12 are designed such that they are capable of binding to the region tags 4 of each of the different reporter oligonucleotides with essentially equal kinetics and binding properties and are optimally designed for use in a single reaction environment with minimal non-specific hybridization.

Once the cRTs 12 have bound the region tags 4 of the reporter oligonucleotides 1, the collecting means/reporter oligonucleotide complexes 130 are analyzed. In the embodiment illustrated in FIG. 2, the collecting means are Luminex microspheres. Prior to analysis the microspheres, along with the reporter oligonucleotides 1, are washed and collected by any convenient means, such as centrifugation. In an alternate embodiment where the collecting means is a silicon chip, prior to analysis, the collecting means 10, along with reporter oligonucleotides 1, is simply washed. The collecting means/reporter oligonucleotide complexes 130 contain the first detection means 6 and the second detection means 14, which generate a first signal and a second signal, respectively, during the analysis. The first and second signals may be different so that the first and second signals are different and can be detected simultaneously in the presence of one another. In the embodiment illustrated in FIG. 2, the first detection means 6 is the fluorescent PE label and the second detection means 14 is the microspheres used in the x-MAP™ technology from Luminex Corporation. The x-MAP™ technology uses a plurality of internally color coded, spectrally addressable polystyrene microspheres. The assigned color-code of the microsphere identifies the reaction throughout the analysis. The microspheres are internally color coded by varying the intensity of two fluorescent dyes. When using 2 fluorescent dyes in this manner over 100 discrete fluorescent signals in the microspheres can be generated. This number can be increased geometrically by increasing the number of fluorescent dyes used. In this embodiment, the first and second signals are discrete fluorescent signals generated when the first and second detection means are excited by laser light as discussed below. As previously mentioned, the first signal indicates that a reporter oligonucleotide has bound its specific target nucleic acid and the second signal serves to identify the specific target nucleic acid sequence. When such microspheres are used as the second detection means 14 the first detection means 6 on each of the reporter oligonucleotides can be the same since the identity of the reaction is determined and tracked by the second detection means.

During the analysis, the collecting means/reporter oligonucleotide complex 130 is analyzed and the amounts of the various reporter oligonucleotides 1 that specifically bound a given target nucleic acid are determined. The analysis involves measurement of 2 signals, the first signal (generated by the first detection means 6) and the second signal (generated by the second detection means 14). Although a variety of first 6 and second 14 detection means may be employed to generate a variety of first and second signals, in the embodiment described, the first detection means is the PE fluorescent label, generating a discrete fluorescent first signal, and the second detection means is the x-MAP microspheres, generating a plurality of discrete fluorescent second signals.

The presence and/or amount of each discrete reporter oligonucleotide that specifically bound its target nucleic acid sequence are determined by the summed intensity of the detected first signals. Controls may be used to quantitate the levels of the targets. The identity of the various reporter oligonucleotides is determined by the detection of the second signal (since each microsphere is specific for a known reporter oligonucleotide 1 by virtue of the cRTs 12). To perform the analysis, the reporter oligonucleotide/collecting means complexes are injected into a detection instrument (such as the Luminex HTS, the Luminex 100 or the Luminex 100IS from Luminex Corporation) that uses microfluidics to align the oligonucleotide/collecting means complexes in single file. A plurality of lasers, in this embodiment 2 lasers, illuminate the complexes producing a first signal from the first detection means and a second signal from the second detection means. The lasers are programmed to illuminate the complexes with the proper wavelength of light to generate the required signals. Next, advanced optics capture the first and second signals. Finally, digital signal processing translates the signals into real-time, quantitative data for each reaction. The result is a determination of the amount of target nucleic acid sequence present in the reaction. As stated above, the present method is uniquely suited to detection of multiple target nucleic acid sequences in one reaction tube since the specificity of the detection step can be separated from the sensitivity of the reaction.

An alternate embodiment of the ROCASH method may be used. The ROCASH method is performed essentially as described in FIG. 2, with the following modifications. After the reporter oligonucleotides 1 bind their target nucleic acid sequence, the unbound reporter oligonucleotides 1 are removed from solution. These unbound reporter oligonucleotides 1 are placed in suitable buffer (such as hybridization buffer) for use in a subsequent round of ROCASH. In the following step, the reporter oligonucleotide/target nucleic acid complex 120 is denatured, and the reverse strands 110 containing the target nucleic acid are removed via the incorporated tag as discussed previously. The target nucleic acids are also placed in a suitable buffer (such as hybridization buffer) for use in a subsequent round of ROCASH. The free reporter oligonucleotide 1 may be analyzed immediately as previously described, or may be collected for pooling with free reporter oligonucleotides 1 collected in subsequent round of ROCASH.

The collected reporter oligonucleotides 1 and the collected reverse strands 110 containing the target nucleic acids are then combined and new round of ROCASH initiated. The procedure can be repeated as many times as desired. In addition, the process may be automated. The subsequent rounds of ROCASH will serve to increase the sensitivity of the reaction. In one embodiment, the ROCASH method may be performed with no or only 1 round of PCR amplification, with the increase in signal intensity being generated by subsequent rounds of PCR amplification of the target nucleic acid

Applications of the ROCASH Method

The ROCASH method may be used for a variety of applications. The applications listed below are illustrative only and are not meant to limit the application of the ROCASH method, but to illustrate its potential for use.

SNP detection. The ROCASH method is well suited for the analysis of single nucleotide polymorphisms (SNPs). SNPs are basically point mutations and are estimated to occur at 1 out of every 1000 bases in the human genome. SNPs may occur in the coding region of genes or may occur in the non-coding portions of the genome. If the SNPs occur in the region of a gene, the structure or function of the encoded protein may be altered, resulting in a disease state. These types of SNPs are commonly analyzed for diagnostic and/or risk assessment purposes. The majority of SNPs appears in the non-coding regions of genes and has no known impact of the phenotype of an individual. However, these SNPs are useful as markers to identify genes that are involved in human diseases. In this use, a collection of SNP's are determined at regular intervals along the human genome. The SNP profile of control individuals is compared to the SNP profile of an individual with a given disease to identify those SNPs that differ between the two individuals. The rationale is that by identifying areas where the SNP profiles are different, these areas are candidate regions for the genetic mutation that causes the disease state. This assumption is made by assuming that the block of different SNPs are inherited along with the genetic mutation causing the disease. Using the ROCASH method, hybridization domains could be designed to recognize each of the allelic variants of the SNP. The hybridization domains are designed such that the presence of a single mismatch between the hybridization domain and the SNP target will reduce the hybridization efficiency such that the reporter oligonucleotide will not bind to the target nucleic acid. As discussed above, each target nucleic acid, in this case an allelic SNP variant, is linked to a known second detection means. Therefore, the identity of an individual SNP, or the determination of a SNP haplotype (a group of SNPs physically linked on the same chromosome within a short distance of each other) can be accomplished quickly and efficiently.

Gene expression profiling. The expression levels of a plurality of genes may also be determined using the ROCASH method. mRNA is isolated using standard techniques. Using an oligo TTTT reverse primer, cDNA first strand strand synthesis is carried out using standard techniques. The oligo TTTT reverse primer may also incorporate a nucleic acid tag means as discussed above. For example, if it is desired to produce quantities of mRNA, it is advantageous to us the T7 polymerase as the nucleic acid tag means. Reporter oligonucleotides designed to hybridize to target sequences in the amplified region may be used as purification means as described above. In an alternate embodiment, no amplification of the DNA is required. The ROCASH method is sentitive enough that given levels of RNA in the 10-20 ug range, genes expressed at moderate levels can be detected. In this embodiment, the endogenous poly A signals on the mRNA are used as the tag means, while oligo TTTT sequences may be used as the purification means as described above. Using the multiplexing capability of ROCASH, the expression levels of multiple genes can be determined.

In addition, controls may be utilized to determine the copy number of the genes of interest. Both internal and external controls may be used. For internal controls, a single gene of known copy number is selected. The internal control is usually a “house keeping” gene that is expressed in every tissue at about the same amount. For external controls, a sequence that is not present in the sample to be tested is selected, such as a sequence from lambda phage. Since the copy number of the external control is known, it can be used to quantitate the expression of the target nucleic acids. A series of reporter oligonucleotides are designed to hybridize to different targets on the control gene, with the hybridization domains being designed to interact with more than one collecting means. For example, if the HPRT gene is selected as the control gene, a series of 14 reporter oligonucleotides may be designed to hybridize to 14 different regions of the gene. Of these 14 reporter oligonucleotides, 2 reporter oligonucleotides have one region tag, 4 reporter oligonucleotides have a second region tag and 8 reporter oligonucleotides have a third region tag. As a result, 2, 4 and 8 of the reporter oligonucleotides will be associated with distinct colors second detection means, such as the Luminex microspheres. The result is a linear graph where signal intensity will be on the y-axis and copy number on the x-axis. By knowing the signal intensity of the gene of interest, its copy number can be determined from this relationship.

Gene dosage mutations. The ROCASH methods may also be used to detect increases or decreases in the copy number of genomic DNA. DNA copy number is associated with many disease states such as, but not limited to, Downs's syndrome, alpha thalassemia, and cancer. In this application, genomic DNA may be subject to standard PCR to amplify a target nucleic acid sequences in the gene of interest and a control gene, and reporter oligonucleotides are designed to hybridize with the target nucleic acid sequences. Preferably the target nucleic acid sequences are selected such that optimal hybridization conditions are compatible for the reporter oligonucleotides. The level of expression of the gene of interest is compared to the level of expression of the control gene to determine the copy number of the gene of interest. Internal controls for gene dosage analysis may be used as is known in the art and described in U.S. Pat. No. 5,888,740.

All references to articles, books, patents, websites and other publications in this disclosure are considered incorporated by reference. The following examples illustrate certain embodiments of the present disclosure without, however, limiting the same thereto.

What is claimed:

1. A method for detecting a nucleic acid comprising
a. providing a nucleic acid source containing at least one target nucleic acid;
b. providing at least one set of reporter oligonucleotides, the reporter oligonucleotides comprising a hybridization domain specific for a known target nucleic acid, a region tag, and a first detection means;
c. contacting the at least one set of reporter oligonucleotide with the nucleic acid source under conditions for high specificity complementary hybridization between the hybridization domain and the target nucleic acid to form at least one reporter oligonucleotide/target nucleic acid complex;
d. separating the at least one reporter oligonucleotide/target nucleic acid complex from unbound reporter oligonucleotide;
e. separating the at least one reporter oligonucleotide/target nucleic acid complex and separating the reporter oligonucleotide from the target nucleic acid;
f. providing at least one set of collecting means comprising a plurality of complementary region tags and a second detection means;
g. contacting the at least one set of collecting means with the reporter oligonucleotide under conditions for high specificity complementary hybridization between the region tag and the complementary region tag; and
h. determining the amount of the reporter oligonucleotide bound the collecting means by analyzing a first signal generated by the first detection means.

2. The method of claim 1 further comprising determining the identity of the target nucleic acid bound by the at least one set of reporter oligonucleotide by examining a second signal generated by the second detection means.

3. The method of claim 2 where the conditions for complementary hybridization between the at least one set of reporter oligonucleotide and the target nucleic acid can be varied independently of the conditions for complementary hybridization between the region tag and the complementary region tag.

4. The method of claim 2 where each hybridization domain is associated with a known region tag and each region tag is capable of hybridizing to a known complementary region tag.

5. The method of claim 2 where each set of collecting means comprises complementary region tags which hybridize only with reporter oligonucleotides specific for a known target nucleic acid so that the known target nucleic acid is associated with a known second signal.

6. The method of claim 2 where each set of collecting means comprises complementary region tags which hybridize to reporter oligonucleotides specific for a plurality of known target nucleic acids so that the plurality of known target nucleic acids are associated with a known second signal

7. The method of claim 6 where each of the multiple target nucleic acids belong to an analysis group.

8. The method of claim 7 where the analysis group is selected from the group consisting of a single gene, a family of related genes and a set of genes involved in a common mechanism of interest.

9. The method of claim 2 where the region tags and the complementary region tags are selected such that a complementary hybridization of the region tags to the complementary region tags will have a Tm that is substantially the same.

10. The method of claim 9 where the region tags display minimal reactivity with sequences of the human genome have at least one of the properties selected from the group consisting of a 60% GC content, at least one CpG dimers, at least one TpA dimer, at least about 20 nucleotides in length, and the complementary region tags have a complementary sequence.

11. The method of claim 2 where the target nucleic acids are amplified from the nucleic acid source prior to contacting the source with the at least one set of reporter oligonucleotide.

12. The method of claim 1 where the target nucleic acids are amplified from the nucleic acid source prior to contacting the source with the at least one set of reporter oligonucleotide such that the amplified target nucleic acids incorporate a tag means and the separating is accomplished by adding a purification means capable of specific interaction with the tag means.

13. The method of claim 12 where the tag means is selected from the group consisting of a first nucleic acid sequence, a first amino acid sequence and a first chemical moiety and the purification means is independently selected from the group consisting of a complementary nucleic acid sequence to the first nucleic acid sequence, a second amino acid sequence complementary to the first amino acid sequence and a second chemical moiety capable of binding to the first chemical moiety, the first nucleic acid sequence or the first amino acid sequence.

14. The method of claim 12 where the tag means is a nucleic acid sequence and the purification means is selected from the group consisting of a nucleic acid sequence complementary to the nucleic acid sequence of the tag means conjugated to a support and a nucleic acid sequence complementary to the nucleic acid sequence of the tag means conjugated to a magnetic bead.

15. The method of claim 12 where the tag means is a biotin moiety and the purification means is selected from the group consisting of a streptavidin moiety conjugated to a support and a streptavidin moiety conjugated to a magnetic bead.

16. The method of claim 2 where the at least one set of collecting means is a spectrally addressable microsphere capable of producing the second signal.

17. The method of claim 16 where each set of microspheres comprises a plurality of complementary region tags which hybridize with reporter oligonucleotides specific for a unique target nucleic acid so that the unique target nucleic acid is associated with a known second signal.

18. The method of claim 17 where first signal is identical for each set of reporter oligonucleotides.

19. The method of claim 18 where the first signal and the second signal are discrete wavelengths of light.

20. The method of claim 19 where the first signal and the second signal are generated by stimulating the first detection means and the second detection means with predetermined wavelengths of light.

21. The method of claim 16 where each set of microspheres comprises complementary region tags which hybridize to reporter oligonucleotides specific for a plurality of known target nucleic acids so that the plurality of known target nucleic acids are associated with a known second signal.

22. The method of claim 21 where each of the multiple target nucleic acids belong to one analysis group.

23. The method of claim 22 where first signal is identical for each set of reporter oligonucleotides.

24. The method of claim 23 where the first signal and the second signal are discrete wavelengths of light.

25. The method of claim 24 where the first signal and the second signal are generated by stimulating the first detection means and the second detection means with predetermined wavelengths of light.

26. The method of claim 22 where the analysis group is selected from the group consisting of a single gene, a family of related genes and a set of genes involved in a common mechanism of interest.

27. The method of claim 26 where the first signal and the second signal are discrete wavelengths of light.

28. The method of claim 16 further comprising an internal control and an external control.

29. The method of claim 2 further comprising an internal control and an external control.

30. The method of claim 21 further comprising an internal control and an external control.

31. The method of claim 2 where the at least one set of collecting means is a silicon chip and the second signal is selected from the group consisting of position, color, electrical properties, chemical properties, physical properties and discrete wavelengths of light.

32. The method of claim 2 where the collecting means is a silicon chip and the second detection means is position

33. The method of claim 32 where the silicon chip comprises a plurality of complementary region tags which hybridize with reporter oligonucleotides specific for a unique target nucleic acid so that the unique target nucleic acid is associated with a known second signal.

34. The method of claim 33 where first signal is identical for each set of reporter oligonucleotides.
